(19) United States
(12) Patent Application Publication
(10) Pub. No.: US 2019/0100727 A1
(43) Pub. Date: Apr. 4, 2019

(54) Cells for Therapy of the Heart

(71) Applicant: Charite Universitatsmedizin Berlin, Berlin (DE)

(72) Inventors: Marion Haag, Berlin (DE); Jochen Ringe, Hohen Neuendorf (DE); Michael Sittinger, Berlin (DE); Carsten Tschope, Berlin (DE)

(73) Assignee: Charite Universitatsmedizin Berlin, Berlin (DE)

(21) Appl. No.: 16/147,775

(22) Filed: Sep. 30, 2018

Related U.S. Application Data
(63) Continuation-in-part of application No. 14/642,816, filed on Mar. 10, 2015, now abandoned.
Application No. 14/642,816 is a division of application No. 12/544,760, filed on Aug. 20, 2009, now abandoned.
Application No. 12/544,760 is a continuation of application No. PCT/EP2008/052027, filed on Aug. 20, 2008.

(30) Foreign Application Priority Data
Aug. 20, 2007 (DE) 10 2007 008 650.6

(51) Int. Cl. C12N 5/077; G01N 33/569; A61K 35/34

(52) U.S. Cl. CPC C12N 5/0657; G01N 33/56966; C12N 2509/00; C12N 2506/1307; C12N 2501/06; C12N 2501/115; C12N 2501/11; A61K 35/34

(57) ABSTRACT

According to the invention fibroblast-like cells obtained from heart muscle biopsies, which are CD90 negative, CD105 positive, CD117 negative and/or CD166 positive as well as cell preparations of such cells for therapy of heart diseases as well as a method for providing the latter are disclosed. The cells according to the invention are characterized by a good cultivability in cell culture. Furthermore a method for obtaining the cells and cell preparations according to the invention are disclosed.

### CROSS-REFERENCE TO RELATED APPLICATIONS

This is a Continuation-in-Part of U.S. patent application Ser. No. 14/642,816, filed Mar. 10, 2015, which is a Division of U.S. patent application Ser. No. 12/544,760, filed Aug. 20, 2009, which was a Continuation of International Patent Application No. PCT/EP2008/052027, filed Aug. 20, 2008, which in turn claimed the benefit of German Patent Application No. 10 2007 008 650.6, filed Feb. 20, 2007. The foregoing patent applications are incorporated by reference herein in their entirety.

### BACKGROUND OF THE INVENTION

The invention relates to cells and a cell preparation for therapy of heart diseases as well as methods for producing the cells and cell preparations according to the invention.

Diseases of the heart and cardiovascular system belong to the most important causes of disease in industrialized societies. Among them, cardiac insufficiency, as a cause or consequence of a pathology caused by other factors, is one of the most common diseases. The number of cases in Europe alone lies within the double-digit millions.

Among experimental therapies, which are discussed with respect to cardiac insufficiency, is also cell therapy. The basis of this therapeutic approach is the expectation that the weakened myocardium should be strengthened by the immigration and the proliferation of the therapeutically applied cells into the myocardium and that its functional efficiency should be increased.

The production of adult stem cells from the heart constitutes a topic of scientific research. Whilst the fundamental ability for regeneration of cardiac tissue has been questioned for a long time, several approaches for regeneration of weakened myocardium either from stem cells or from not fully differentiated stem cell-like cells are presently under examination and in part under clinical development.

Pittenger et al. have characterized mesenchymal stem cells (Science 284, 143-147); which show inter alia a CD90 positive phenotype.

Wang et al. (International Journal of Cardiology 109 (2006) 74-81) showed the differentiation of mesenchymal stem cells of the rat to differentiated heart cells in co-culture with fully differentiated heart cells. The cells obtained thereby are also CD90 positive. Similar results are found by Moscoso et al. (Transplantation Proceedings, 37, 481-482 (2005)) in porcine cells.

Messina et al. (Circulation Research, Oct. 29, 2004, pp. 911-924; WO2005/012510) describe a method for isolation and cultivation of heart cells from biopsies. The cells isolated there are inter alia c-kit/CD117 positive.

### SUMMARY OF THE INVENTION

A fundamental problem in the application of cell therapy for the therapy of heart diseases is to obtain sufficient amounts of cells for therapeutic application.

The problem underlying the present invention is to obtain, in a simple method from cell material that is relatively easily accessible outside the body, a cell preparation which is suitable to be applied to a patient suffering from cardiomyopathy and other heart diseases, for instance infarcts and their aftereffects, with the aim of improving patients' cardiac output.

According to the invention, this problem is solved by an isolated mammalian cell having the features: the cell is a cell that was proliferated in cell culture from a primary culture of a tissue sample obtained from a mammal; the cell is a fibroblast-like cell; the cell CD90 negative; CD105 positive; CD117 negative, CD106 (VCAM1) negative. Microarray profiling shows an expression of ACTC1, POP3, VE-Cadherin (CDH5), CDH13, and the lack of expression of NPNT and TERT, so cells show no telomerase reverse transcriptase activity.

Further, this problem is solved by a method for obtaining a cell preparation, wherein

a) a tissue sample obtained from mammalian heart muscle tissue is cultivated in a first cell culture step under conditions suitable for the culture of mammalian cells in cell culture medium in a cell culture container having a solid surface,

b) cells grown out of the tissue sample adhering to the solid surface are detached in a passage step by means of limited proteolysis, isolated and are cultured again, diluted in a cell culture medium,

c) CD90 positive cells are excluded from the detached cells via magnetic activated cell sorting or fluorescence activated cell sorting in a CD90-sorting step,

d) the sorted cells are cultured.

Further, this problem is solved by a cell preparation comprising the cells according to the invention and by a cell preparation which can be generated by a method according to the invention.

### BRIEF DESCRIPTION OF THE DRAWINGS

FIG. 1A shows biopsy material in a cell culture container and a centimeter scale laid underneath the container.

FIG. 1B shows in A-E schematic cell shapes, which may comprise the fibroblast-like cells according to the invention. F and G show cell shapes, which differ from the fibroblast-like cells according to the invention.

FIG. 2 shows morphology and FACS analysis of the cells or cell preparations according to the invention. First row: Left panel: Adherent cells grown in culture (after 5 days in culture), middle panel (after 15 days in culture), right panel, cell culture of the harvested cells in passage 4 after 5 days in culture. Second and third rows: Histograms of FACS analysis, namely: forward versus sideward stray light, FACS analysis for CD90, CD117, CD105 and CD73 is shown as labelled.

FIGS. 3 and 4 show examples of the growth kinetics of example cultures.

FIG. 5 shows the evaluation of an experiment for mixed lymphocyte reaction of 5 patients (n=5).

FIG. 6 shows the results of the FACS sorting: top from the left to the right: unsorted, CD90 positive cells; CD90 negative cells.

FIG. 7 shows the surface marker profile of atrial-appendage-derived cells. Top Left panel: Magnetic cell separation (MACS) was performed to eliminate CD90+ cells to be left with a CD90low population of atrial appendage-derived cells. The expression values of CD90 surface marker before and after sorting are shown for 7 different donors. Top Right panel: Mean expression of a defined marker set from 7 donors: CD29+, CD44+, CD45−, CD73+, CD90low, CD105+, CD166+. Error bars are shown as SEM. Lower panel: Typical CardAP profile in flow cytometry analysis generated by measuring CD90low atrial appendage-derived cells from one donor. Unstained cells served as negative control and are shown as an overlaid black line.

FIG. 8 shows the VEGF and IL-8 secretion by atrial appendage-derived cells. Mean VEGF (upper panel) and IL-8 (lower panel) levels in passage 3, 4 and 5 per 10⁵ cells. Bar graphs show SEM. No significant difference in secretion could be detected between the three passages.

FIGS. 9A and 9B show the hierarchical cluster analysis of CD90low atrial appendage-derived cells and EMB-derived CardAP cells. Hierarchical cluster analysis of all genes annotated with GO term “angiogenesis” (FIG. 9A) and GO terms “vasculature development”, “blood vessel development” and “blood vessel morphology” (FIG. 9B). EMB-derived CardAP cells (EMB 1-3) and atrial-appendage-derived cells (AA 1-3) cluster in two main groups, while gene expression among all 6 GeneChips is very similar.

FIG. 10 shows the growth kinetics and estimation of cell numbers for therapeutic use. Upper panel: Doubling time of atrial appendage-derived cells from 5 different donors stays relatively constant over the first 5 passages. Lower panel: Theoretical mean cell count calculated for 1 atrial appendage. After passage 3, 30 patients could be treated with an assumed dose rate of 109 cells and 286 patients in passage 4.

### DETAILED DESCRIPTION

According to one embodiment of the present invention a cell or a preparation of a plurality of cells can be provided for therapy of heart disease by bringing biopsy material obtained from a patient or a tissue sample from cells of a patient into a primary culture and proliferation through several passages. Obtaining cells from a primary culture, which was established from a heart muscle biopsy, for instance from a pinhead-sized or even smaller biopsy sample, is preferred. The biopsy materials are “plucked” by means of a small “caliper”. The culture conditions are described in example 1.

According to an embodiment of the present invention the method is distinguished from the one described by Messina at al. (see above) by the absence of cardiotrophin and thrombin in the cell culture medium.

Furthermore, no 2-mercaptoethanol is used in the medium. According to one embodiment of the present invention, the cells grown out of biopsy material are trypsinized. Thus, all steps described by Messina et al. for “harvesting” cells are omitted. The cells are not proliferated in coated cell culture dishes, since these cells adhere very well. Further cultivating of the cells is thus also distinguished, since the “cardiospheres” described in Messina et al. do not adhere, i.e., they do not grow on solid surfaces in culture, such as for instance the bottom of a cell culture vessel, a wall of a cell culture bottle, a film in a nutrient medium or a porous matrix for cell culture. Preferred cells of the present invention do adhere.

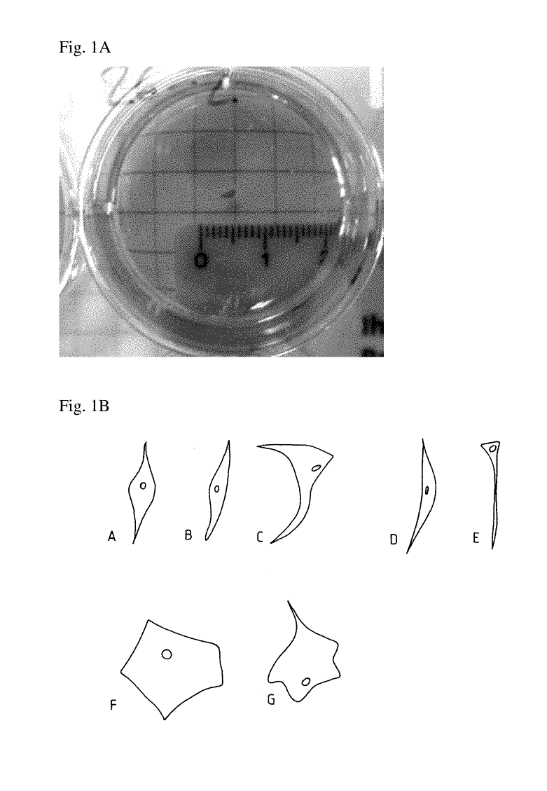

According to one embodiment of the present invention, a method is provided, which allows obtaining a cell population that is characterized by a series of specific characteristics: the cells are fibroblast-like, i.e. elongated, spindle-shaped cells having a morphology as shown in FIG. 1B), and they grow adherently under cell culture conditions shown in example 1.

Surprisingly, these cells are negative with respect to staining by the marker CD90 characteristic for mesenchymal stem cells and fibroblasts.

In this context it is apparent that no homogeneous cell population can be obtained by creating, for example, a primary culture from a heart biopsy material. It is also apparent that for therapeutic application, the application of a plurality of cells, i.e., a cell preparation, would currently be considered. Such cell preparation can be obtained by a cell culture method as described in example 1. The cells contained therein will differ with respect to their phenotype, since they are not grown from a homogeneous population as described above. Accordingly, the characteristic features of a cell preparation according to the invention are not be stated as absolutely “positive” or “negative” concerning a cell marker, but with respect to the whole cell population.

Therefore, according to one embodiment of the present invention, the cell preparation can be characterized in that a plurality of the cells contained therein is CD90 negative. This means that at least 50% of the cells contained in this population are not stained any more by a standard dye marker in the FACS, for instance the dye marker stated in the examples, as cells typically known to the person skilled in the art as CD90 negative cells. CD90 (Thy-1) is a marker for thymus cells, hematopoietic stem cells, NK cells and endothelial cells, fibroblasts and myofibroblasts. Preferably, according to an embodiment of the present invention, the cell preparation is at least 80%, more preferably 90% CD90 negative. Even more preferred are cell preparations which are more than 95%, 98% or 99% CD90 negative.

In order to achieve a preferred homogeneity with respect to CD90 negativity of the cell preparation, the method for obtaining the cell preparation can comprise according to one embodiment of the present invention a purification step, in which the cells are selected with respect to their expression of CD90. For this purpose, methods of fluorescence-based cell sorting (FACS sorting) in suitable devices are known for example. Thereby, cells marked by a fluorescence-marked antibody against the respective antigen are automatically separated in a capillary into a negative and a positive population.

Furthermore, separation by means of magnetic separation is known. Thereby, cells are separated in a very strong magnetic field by means of retention of the antigen positive cells by antibody-coupled magnetic particles.

Means and methods for separation of cells regarding their expression of antigens are known to experts. According to an embodiment of the present invention the cell preparation may also be obtained using a method which, as an alternative or in addition to the separation with respect to CD90 negativity, comprises further separation steps, which select regarding further possible characteristics of the cell preparation:

According to an embodiment of the present invention the cell preparation can be characterized in that a plurality of the cells contained therein is CD105 positive. This means that at least 50% of the cells contained in this population are stained in the FACS by means of a standard dye marker, for instance the dye marker stated in the examples. CD105 is a typical marker for endothelial cells and mesenchymal cells. Preferably, according to an embodiment of the present invention, the cell preparation is at least 90%, more preferably at least 98% CD105 positive.

Furthermore, according to an embodiment of the present invention, the cell preparation is characterized in that a majority of the cells contained therein is CD117 negative. This means that at least 50% of the cells contained in this population are not stained anymore by a standard dye marker in the FACS, for example the dye marker stated in the examples, as cells typically known to the person skilled in the art as CD117 negative cells (negative test). CD117 (c-kit) is a stem cell marker. Preferably, according to an embodiment of the present invention, the cell preparation is 60%, more preferably at least 70% CD117 negative.

Also, according to an embodiment of the present invention, the cell preparation can be characterized in that the majority of cells contained therein are CD166 positive. This means that at least 50% of the cells contained in this population are stained by a standard dye marker, for example the dye marker stated in the examples, in the FACS. CD166 is the acronym for “activated leukocyte cell adhesion molecule (ALCAM)”, a marker typical for mesenchymal stem cells from the bone marrow. Preferably, according to an embodiment of the present invention, the cell preparation is at least 60%, more preferably at least 70% CD166 positive.

Furthermore, according to an embodiment of the present invention, the cell preparation can be characterized in that a majority of the cells contained therein is CD34 negative and CD45 negative. This means that at least 50% of the cells contained in this population are not stained anymore in the FACS by a standard dye marker, for example the dye marker stated in the examples, as cells typically known to the person skilled in the art as CD34-negative or CD45-negative, respectively (negative test). CD34 and CD45 are both stem cell markers. Preferably, the cell preparation is at least 60%, more preferably at least 70% CD34 and CD45 negative.

Furthermore, according to an embodiment of the present invention, the cell preparation can be characterized in that the majority of the cells contained therein is desmin positive after myogenic induction (see examples), and/or positive with respect to antibodies against cardiac smooth muscle myosin. Preferably, the cell preparation is at least 60%, more preferably at least 70% desmin positive and/or myosin positive.

Besides the stated characterization as CD90 negative, the cell preparations may comprise according to an embodiment of the present invention all stated characteristics concerning the fraction of CD105 positive, CD117 negative, CD166 positive, CD34/35 negative cells.

In this respect, the statement of “positivity” or “negativity” can be related to two different populations. This shall be explained by means of a cell preparation denoted as “60% CD105 positive, 90% CD90 negative”:
On one hand, this denotation can relate to a cell preparation in which 60% of all cells are CD105 positive. In the same way the characterization 90% CD90 negative would relate to the whole population. Since here the definition of positivity or negativity does not relate to the whole population, respectively, this criterion shall be denoted as “global characterization”.
On the other hand, a cell preparation in which 60% of the CD90 negative cells are CD105 positive, can be described in this way. This characterization, in case of which a second criterion is only applied to cells which fulfill the first criterion, shall be denoted here as “cumulative characterization”.

The amount of cells contained in a cell preparation, which is definitely CD90 negative and CD105 positive, may possibly differ depending on whether the global or the cumulative characterization is used as a definition. This difference will be larger the more criteria are considered, especially in the case of criteria near 50%.

According to an embodiment of the present invention, a cell preparation shall be defined by means of the stated criteria with respect to expression of CD90, CD105, CD117, CD166 and CD34/45 in a global as well as cumulative characterization, namely by means of stated percentages, wherein the cumulative characterization is preferred.

The method allows for the first time to obtain cell preparations having a cell count of more than 10¹⁰, actually up to 10¹³⁻¹⁴ cells for therapeutic application to the heart.

According to the invention, cells can be administered to the patient as a pharmaceutical preparation. At first, it is preferred in this connection, to administer autologous cell preparations to human patients. Known immunological problems contravene the application of heterologous cell preparations; there are however realistic indications, in case of which even heterologous cell preparations are to be preferred to, for instance, the transplantation of a donor heart. Particularly, the cell preparation offers the great advantage that, for example, cell surface proteins, which allow the identification of the heterologous cells by the patients' immune system, can be concertedly blocked during the preparation method, so that the heterologous cell preparation does not comprise or comprises significantly reduced the known immunological disadvantages of a heterologous transplant.

For the application of the cells according to the invention a plurality of known methods exists. Gyöngyösi et al. describe inter alia (J. Kardiol 2004, 11 (Supp B; pp 22-24) the direct injection of cells into the heart muscle by means of an intra-myocardial catheter-based injection. Since cells comprise tropism, a systemic application is further conceivable.

### DEFINITIONS

Cells in the sense of this invention are somatic cells of mammals, particularly humans. According to the invention, cells and cell preparations in accordance with the independent claims can be used for preparation of a pharmaceuticals for disease therapy. In this respect, the preparation of an autologous cell or cell preparation as well as of an allogeneic cell or cell preparation is possible. Autologous cells or cell preparations are obtained by taking biopsy material from the same patient to whom they are returned. Allogeneic cells or cell preparations are obtained from a different person.

Cells or cell preparations can also be obtained from biopsy material taken from a donor heart. These cells may then be extracorporeally proliferated and stored, in order to be given to the transplanted patient in case of a medical necessity.

Denoting a cell population as “fibroblast-like” in the sense of the present invention means that the majority of the cells display a substantially spindle-shaped appearance under the microscope. Spindle-shaped means that the majority of the cells, according to the invention, comprise an elongated shape, for instance that the cells at confluence have a length of 150-250 μm. At lower confluence however, one also finds cells having a length of merely 60 μm up to over 350 μm (elongated shape). The width of the cells can lie within the range of 13-20 μm, wherein 9 μm and more than 30 μm are also possible.

The cells are also distinguished by their characteristic shape as shown in FIG. 1B) (A-E). Furthermore, fibroblast-like cells in the sense of the present invention are characterized in that they stick or adhere in culture to the bottom of the cell culture container according to the cell culture conditions stated in the examples and can be detached from the bottom of the cell culture container by means of trypsinization. The more confluent (dense) the cell culture, the more uniformly fibroblast-like the cell shape.

The denotation of a cell as “negative” in relation to a tissue marker, such as for example a protein of the CD (“cluster of differentiation”)-series, e.g. CD90, CD105, CD117, CD166, means that the cell is not stained by means of a prescribed staining with a marked antibody against the denoting marker in a way, that the cell yields concerning the order of magnitude a comparably strong signal with respect to the labeling of the respective antibody in the FACS (fluorescence-based cell sorting device) or fluorescent microscopy as a cell that is regarded by the experts as “positive” in relation to the respective surface marker with the same antibody and under comparable staining conditions.

The same holds true in an analogous fashion for staining methods inside the cell.

Known, unambiguous examples for cell types denoted as positive or negative concerning two substantial markers in relation to the present invention are mesenchymal stem cells. These are positive for CD 105 and CD 90 and negative for CD 34 and 45.

According to an embodiment of the present invention a primary tissue sample obtained from a living organism or an organism dead for less than 24 h is brought into culture in a nutrient medium in a cell culture container in a suitable manner as known to a person skilled in the art, for example at 37° Celsius, 95% humidity and 5% CO2. This culturing step includes a partial digestion of the tissue sample by means of proteases. Preferred in this connection are trypsin-EDTA and collagenase IV. Alternatively, the digestion may be conducted without trypsin-EDTA.

This culture is denoted as “primary culture”. During growth cells are observed at regular intervals and harvested/isolated after reaching a predefined cell density. According to an embodiment of the present invention, cells grow out of the biopsy material (outgrowth culture) and are then harvested or isolated. These cells are in turn cultured and passaged each time the cells cover 70-90% of the bottom of the cell culture container (confluence of 70-90%).

Transferring cells from one culture container to another, wherein most of the times a dilution of the cells occurs, is denoted as a passage. This term is a synonym for sub culture and should not be confused with the passage in virology (Toni Lindl, “Cell- and tissue culture” 4^{th} edition, Spektrum Verlag, p. 255). Typically, the cells are thereby detached from the bottom of the cell culture container by applying trypsin (“trypsinization”) and are sown again at a density of 5000-6000 cells/cm². Thus, the cells are transferred from one passage into the next passage.

The highest density arrangement of adherent cells possible as a mono layer in culture is denoted as “confluence” (see also Lindl, ibid., p. 253).

Diseases which can be treated with the cells, cell preparations and pharmaceuticals of the independent claims or indications for the application of the inventive cells and cell preparations may be inter alia heart diseases such as the ischemic cardiomyopathy with good and bad ejection fraction, inflammatory cardiomyopathy with good and bad ejection fraction, diastolic dysfunction, aortic valve defects (stenosis, insufficiency), mitral valve defects (stenosis, insufficiency), right heart insufficiency, bradycardiac and tachycardiac dysrhythmia including AV blocks and atrial fibrillation, coating of coronary stents, diabetic cardiopathy, collagenosis having cardiac involvement, familiar cardiomyopathies, virally induced myocarditis and cor hypertensivum.

According to an embodiment of the present invention, an isolated mammalian cell is provided which is characterized by the following features:
the cell is a cell proliferated in cell culture from a primary culture of a tissue sample obtained from a mammal,
the cell is a fibroblast-like cell, and
the cell is CD90 negative.

Preferably, the cell was proliferated by means of at least three passages in cell culture. The cell can be of human origin.

According to a preferred embodiment, the isolated mammalian cell is CD105 positive. According to a preferred embodiment the isolated mammalian cell is CD105 positive, CD117 negative, CD166 positive, CD34/45 negative, CD106 (VCAM1) negative. According to a preferred embodiment, the cell is characterized by an expression of ACTC1, POP3, VE-Cadherin (CDH5), CDH13, and the lack of expression of NPNT, and TERT. With the lack of TERT expression, the cells have no telomerase reverse transcriptase activity. Expression may be determined by microarray profiling.

Furthermore, a method for obtaining a cell preparation is provided, in which
a) in a first cell culture step, a tissue sample obtained from mammalian heart muscle tissue is cultured under conditions suitable for culture of mammalian cells in cell culture medium, in a cell culture container having a solid surface;
b) cells are detached that have grown out of the tissue sample and that adhere to the solid surface in a passage step by means of limited proteolysis, the detached cells are isolated;
c) the sorted cells are cultured in a second cell culture step.

According to a preferred embodiment, said fibroblast-like cells are characterized by
a) an expression of one or more genes selected from ACTC1, POP3, VE-Cadherin (CDH5) and CDH13 and/or
b) a lack of expression of NPNT and/or TERT.

According to a preferred embodiment, said fibroblast-like cells are characterized by expression of ACTC1, POP3, VE-Cadherin (CDH5) and CDH13 and by lack of expression of NPNT and TERT. With the lack of TERT expression, the cells have no telomerase reverse transcriptase activity.

According to a preferred embodiment, the tissue sample obtained in the method contains heart muscle tissue. According to a preferred embodiment, said tissue sample comprises or essentially consists of endomyocardial cells. According to a preferred embodiment, said tissue sample comprises or essentially consists of atrial appendage cells, and subsequent to said passage step, CD90 positive cells are excluded from the detached cells via magnetic activated cell sorting or fluorescence activated cell sorting in a CD90-sorting step.

According to a preferred embodiment, the tissue sample is digested, prior to the first cell culture step, by one or more connective tissue digesting enzymes. More preferably, the connective tissue digesting enzyme activity is trypsin-EDTA or collagenase IV or a combination of both activities and the duration of the time limited digestion is less than 10 minutes at an activity of 0.05 to 0.25 u/500 ml for trypsin and/or 0.2 to 4.5 units/ml [u/ml] for collagenase IV.

According to a preferred embodiment of the method, the first cell culture step has a duration of 7 to 15 days. It is further preferred, that the passage step is conducted at a confluence of the cells adhering to the solid surface of 70% or larger, and/or that the cell culture medium of the first cell culture step or the passage step or of both steps does not contain cardiotrophin, thrombin or mercaptoethanol.

According to a preferred embodiment of the method, the cell preparation obtained in the previously described steps a to c is subjected to a step of myogenic induction, as a consequence of which cells positive for staining with α-desmin and/or myosin antibodies can be obtained.

According to a preferred embodiment of the method, the cell preparation obtained in the steps a to c is subjected to a purification step, in which:
the cells contained in the cell preparation are brought into contact with molecules capable of binding specific cell surface markers and
those cells, to which the molecules capable of binding specific cell surface markers have bound, are separated.

Thereby, the molecules capable of binding specific cell surface markers can be antibodies against CD90, CD105, CD117, CD166, CD34 and/or CD45.

In the purification step, molecules capable of binding specific cell surface markers can be bound to magnetic particles and retained during the purification step in a magnetic field. The cells may also be separated by means of fluorescence-activated cell sorting (FACS).

Furthermore, a cell preparation is provided that contains cells that are CD105 positive, CD117 negative, CD166 positive, CD34/45 negative, α-desmin positive and/or myosin positive.

Furthermore, a cell preparation obtained by means of the afore-characterized purification step is provided. Preferably, the cells contained therein are more than 90% CD90 negative, more than 90% CD105 positive and more than 50% CD117 negative. More preferably, the cells contained therein are more than 95% CD90 negative, more than 95% CD105 positive, more than 60% CD117 negative and more than 50% CD166 positive. Furthermore, the cells in the cell preparation characterized in this way may be more than 90% CD34 negative and more than 90% CD45 negative.

Furthermore, a cell preparation is provided, in which the cells contained therein are more than 95% CD90 negative, and more than 90% of the CD90 negative fraction of the cell preparation consists of CD105 positive cells. More preferably, the cells contained therein are more than 95% CD90 negative, and more than 90% of the CD90 negative fraction of the cell preparation is CD105 positive and more than 50% of the CD90 negative fraction is CD117 negative. More preferably, the cells contained therein are more than 98% CD90 negative, and the CD90 negative fraction of the cell preparation is more than 95% CD105 positive, more than 60% CD117 negative and more than 50% CD166 positive.

Furthermore, a cell preparation is preferred in case of which the cells contained therein are more than 95% CD90 negative, and the CD90 negative fraction of the cell population is more than 90% CD105 positive and the fraction that is both CD90 negative and CD105 positive is more than 60% CD117 negative. Furthermore, a cell preparation is preferred in case of which the cells contained therein are more than 90% negative for CD106 (VCAM1). Furthermore, a cell preparation is preferred in case of which transcripts therein were detected for expression of ACTC1. Furthermore, a cell preparation is preferred in case of which transcripts therein were detected for expression of POPS. Furthermore, a cell preparation is preferred in case of which transcripts therein were detected for expression of VE-Cadherin (CDH5).

Furthermore, a cell preparation is preferred in case of which transcripts therein were detected for expression of CDH13. Furthermore, a cell preparation is preferred in case of which the cells therein lack the expression for NPNT. Furthermore, a cell preparation is preferred in case of which the cells therein lack the expression for TERT.

The afore-characterized cells or cell preparations can be used for producing a pharmaceutical for therapy of heart diseases, particularly cardiomyopathy. In order to further illustrate the present invention and the advantages thereof, the following specific examples are given, it being understood that same are intended only as illustrative and in nowise limitative.

### EXAMPLES

### Materials and Methods

### Atrial Appendages

Atrial appendages were obtained from 7 patients undergoing heart surgery at the Deutsches Herzzentrum Berlin (DHZB). Right atrial appendages were resected on the beating heart, immediately prior to venous cannulation. All patients provided written informed consent to participate in this study. Donation of cardiac tissue was approved by the local ethical committee of the Charite Universitatsmedizin Berlin (No. 4/028/12).

### Isolation of Atrial Appendage-derived Cells

Appendages were washed with phosphate buffered saline (PBS; Biochrom, Berlin, Germany) and fragments of ˜1 mm3 were cut from the myocardium using a sterile scalpel and tweezers and then fixed to the bottom of 6-well-plates. Outgrowth cultures were performed in Iscove's Modified Dulbecco's Medium (IMDM; Biochrom) containing 10% allogenic serum (German Red Cross, Berlin, Germany) and 1% penicillin/streptomycin (Biochrom). Fragments were cultured under standard culture conditions and the medium was partly replaced every 2-3 days. Cell harvest was performed after about 13 days. Outgrowing cells were washed with PBS and trypsinized with 0.05% trypsin/0.02% EDTA (Biochrom) and then subjected to immunomagnetic sorting with microbeads (MACS; human CD90 MicroBeads kit, Miltenyi Biotec, Bergisch Gladbach, Germany) to obtain a CD90^{low} cell population. Therefore, harvested cells were counted and 320 PI MACS buffer (PBS/0.5% BSA/0.4% EDTA) and 80 PI of Anti-CD90 microbeads per 5×10⁶ cells were added and left for 1 hour of incubation in the dark at 4° C. Then, cells were washed and filtered first on LS MACS columns and in a second step on LD MACS columns. The eluate contained the CD90^{low} cell population that was used in all experiments with atrial appendage-derived cells. CD90^{low} cells were seeded in cell culture flasks at a density of 6,000 cells/cm2 and expanded using cIDH medium consisting of IMDM, Dulbecco's Modified Eagle Medium (DMEM) and Ham's F12 medium (all Biochrom) in equal amounts, and supplemented with 5% allogenic serum (German Red Cross), 1% penicillin/streptomycin (Biochrom), 100 ng/ml basic fibroblast growth factor (bFGF; Peprotech, Hamburg, Germany) and 100 ng/PI epithelial growth factor (EGF, Peprotech). Cells were cultured under standard culture conditions and replacement of medium was conducted every 2-3 days.

### Immunofluorescence Staining of Atrial Appendage-derived Cells

CD90^{low} atrial appendage-derived cells were seeded on LabTek chamberslides (Becton Dickinson, Heidelberg, Germany). Normal human dermal fibroblasts (NHDF) served as a positive control. Cells were fixed with 4% formalin (Herbeta, Berlin, Germany) for 10 minutes and washed with PBS. Blocking was performed for 1 h using 5% donkey serum (Sigma-Aldrich, Taufkirchen, Germany) and 1% BSA (Sigma-Aldrich) diluted in PBS. CD90 antibody (Acris, Herford, Germany) was applied at a 1:100 dilution in donkey blocking buffer and incubated overnight. After washing with donkey blocking buffer, an Alexa488 coupled anti-mouse antibody (Abcam, Cambridge, UK) was applied at a 1:50 dilution and incubated for 90 minutes at room temperature. Nuclei were stained with a 1:400 bisbenzimide/BSA solution (Hoechst, Frankfurt a. M., Germany). Images were obtained on a Zeiss AxioObserver microscope. Contrast was adjusted in the same matter to all samples using Zeiss AxioVision software.

### Flow Cytometry Analysis

Flow cytometry analysis was carried out on a FACSCalibur cytometer (Becton Dickinson). Staining was done with fluorescein isothiocyanate (FITC) labeled mouse anti-human CD90 before and after sorting. Additionally, cells in passage 4 were stained with mouse anti-human CD44, CD45 and CD105 (also FITC labeled), and CD29, CD73 and CD166 (phycoerythrin; PE labeled) antibodies. CD105 antibodies were purchased from Acris, the others from BD Pharmingen (Heidelberg, Germany). After being trypsinized, for each antibody staining 2.5×105 cells were incubated for 15 min on ice in the dark. Cells were washed before analysis. Additional staining with propidium iodide (Sigma-Aldrich) was carried out to then exclude apoptotic cells and cell debris. Unstained cells served as negative control. CellQuest software (Becton Dickinson) was used to acquire and evaluate the data.

### Enzyme-linked Immunosorbent Assays

In passage 3, 4 and 5, CD90^{low} atrial appendage-derived cells were seeded with a density of 6 000 cells/cm2. 24 h later, supernatants were collected and stored at −20° C. Cells were harvested and counted to calculate the amount of secreted IL-8 and VEGF per 105 cells. Supernatants from 4 donors were thawed and used in ELISA assays (R&D Systems, Wiesbaden, Germany) according to the manufacturer's protocol. Concentration of vascular endothelial growth factor (VEGF) and interleukin-8 (IL-8) was measured using a photometric microplate reader (Tecan, Crailsheim, Germany).

### RNA Isolation and Genome-wide Microarray Analysis

Passage 4 CD90^{low} atrial appendage-derived cells from 3 donors were lysed using TRI reagent (Sigma-Aldrich) and RNA was isolated using the RNeasy Mini Kit (Qiagen, Hilden, Germany). Briefly, frozen cell lysates of 1 ml were thawed and then mixed with 133 PI of 1-bromo-3-chloro-propane (Sigma-Aldrich) to be followed by 15 minutes at 10 g in a thermomixer and afterwards 60 minutes of centrifugation at 16 000 g. The aqueous phase was collected and the RNA precipitated using 70% ethanol. The lysate was further purified according to the manufacturer's protocol. Agilent Bioanalyzer and NanoDrop spectrophotometer (Agilent, Santa Clara, Calif., USA) were used to analyze integrity and purity of the samples.

### Gene Expression Profiling Analysis

Gene expression profiling was carried out with Affymetrix HG-133 plus 2 GeneChips (Affymetrix, Santa Clara, USA). The data were analyzed according to Affymetrix recommendations. In brief, 250 ng of RNA were used to synthesize biotin-labeled cRNA and 10 Pg of fragmented cRNA were then hybridized to GeneChips for 16 h at 45° C. Affymetrix equipment was used for washing, staining and scanning. Raw data were processed and normalized using Affymetrix GeneChip Operating Software (GCOS) 1.4.

### Microarray Data Analysis Strategy

Expression data from CD90^{low} appendage-derived cells of 3 different donors were screened, and compared to already published EMB-derived CardAP cell data of 3 different donors (Haag et al., 2010). Generally, we were interested in expression of genes involved in angiogenesis and vasculogenesis. A second aim was to compare atrial appendage-derived cells to formerly described EMB-derived CardAP cells, assuming strong similarities due to the same isolation protocol. In a first step, we wanted to get insights in whether CD90^{low} atrial appendage-derived cells would have pro-angiogenic potential on the gene level and if so, what are the genes that might be interesting to further look into for future research. To do so, the inventors screened for 84 key genes commonly used in angiogenesis PCR arrays (van Beem et al., 2008, Wang et al., 2010). The inventors were only interested in genes whose expression was detected as present (p<0.05) in all three donors as at least 1 probe set ID. The same procedure was applied to formerly acquired micro array data from EMB-derived CardAP cells. In a second step, the inventors were interested in genes whose expression was significantly (p<0.05) up- or downregulated. Hence, each of the 3 GeneChips of the EMB-derived CardAP cell group was compared to each of the 3 GeneChips of the atrial appendage-derived cell group. Differentially expression was assumed when a fold change of ≤2 or ≥2 occurred in more than 80% of the nine comparisons. The selected genes were uploaded in the Database for Annotation, Visualization and Integrated Discovery (DAVID) 6.7 (Huang da., Sherman, & Lempicki, 2009, Nat Protoc, 4: 44-57) and screened for their involvement in biological processes with focus on angiogenesis, blood vessel, vasculature and heart/cardiac development. Annotations within DAVID were based on the three gene ontology databases classified by biological process (GOTERM_BP_FAT), cellular component (GOTERM_CC_FAT) and molecular function (GOTERM_MF_FAT).

Classification of Genes into Clusters

The inventors hypothesized that albeit showing differentially expressed genes, atrial appendage-derived cells would cluster similar to EMB-derived CardAP cells in functional categories related to tissue regeneration. To verify this hypothesis, two groups were formed as follows: Group 1 contained all genes annotated with the GO term “angiogenesis”, Group 2 all genes annotated with “blood vessel development” “blood vessel morphogenesis” and “vasculature development”. Only genes with 100% detection (p<0.05) were selected. If multiple variants of a gene were 100% present, all but one were eliminated. Gene Expression Similarity Investigation Suite (GENESIS) 1.7.7 software (Sturn, Quackenbush & Trajanoski, 2002, Bioinformatics, 18: 207-8) was used for hierarchical clustering. Expression values of all 6 donors in each group were log2 transformed. Experiments were normalized. Clustering was done as complete linkage for experiments and genes.

### Evaluation of Growth Kinetics

CD90^{low} atrial appendage-derived cells isolated from 5 different donors were seeded in passage 2 at a density of 6 000 cells/cm² in 25 cm² cell culture flasks (3 per donor), cultured for 5 to 6 days, trypzinized, counted and plated again at the initial cell density. The procedure was continued until the cell number was less then needed to plate them again. To determine the growth kinetics, since not all harvested cells were plated again, the theoretical cell number (N) was calculated using the equation N=N0×ePt where N0 represents the cell number at t=0, and P the growth rate. The cell doubling time (td) was determined using the equation td=In 2/P.

### Estimation of Maximal Cell Number per Atrial Appendage

Atrial appendages from 3 donors were used to determine the mean number of tissue fragments that could be obtained from an appendage in general. The appendages were weighed and processed as described above. The total number of tissue fragments were counted. 126 fragments were used for outgrowth cultures, placing 3 fragments in one well of a 6-well plate. Cell harvest, pooling of all cells and immunomagnetic sorting was performed after 13 days. The results of the subsequent cell counting and of the growth kinetics studies were used to calculate the theoretical number of CD90^{low} cells in passage 3 and 4 using the equation N=N0×ePt.

### Statistical Analysis

Statistical analysis and drawing graphs was performed with GraphPad Prism 7.0a (Graphpad Software, La Jolla, USA). For comparisons of two groups, student t-test was used. For three or more group comparisons one-way ANOVA was performed. Data sets are reported as means +/− standard error of the mean (SEM) and asterisks were assigned to the p-values in the order p***,0.001, p**,0.01 and p*,0.05 for statistical significance.

### Example 1

### Culture Conditions and Passage

### Reprocessing of Biopsy Materials:

Biopsy material obtained from a heart muscle was cut into pieces up to 5 mm³ in size, preferably 1-2 mm³ with a sterile scalpel and washed with PBS (phosphate-buffered isotonic solution of sodium chloride) (free of calcium and magnesium).

The tissue samples obtained in this way were digested 3×5 min at 37° C. with Trypsin/EDTA and 0.45 u/ml collagenase IV (Sigma Aldrich) in PBS (1:500 diluted, activity of the undiluted solution 0.125-0.15 u/ml; Biochrom AG, Berlin). After 5 minutes, the biopsy materials were transferred into a new trypsin-collagenase mixture, respectively. The supernatant was discarded and the pre-digested tissue was washed with IMDM (Iscove's Modified Dulbecco's Medium completed with 10% FBS (fetal bovine serum), 100 u/ml Penicillin, 100 μg/ml streptomycin, 2 mmol/L L-glutamine), afterwards the explants were cultivated in completed IMDM medium in a cell culture container having 9.6 cm² growth surface. Explants have to be fastened (“firmly pressed”) to the bottom of the culture container.

Depending on the explant (depending on the individual patient) fibroblast-like, adherent cells grow out after 7-15 days.

### Harvest of Grown Cells:

The explants/cells were carefully washed with PBS (explants should not detach), and then incubated for 3-5 minutes with 1 ml Trypsin/EDTA (0.05%/0.02%) per explant (culture panel size 9.6 cm², see above), and afterwards the digestion reaction was stopped with IDH medium (IMDM, DMEM, Ham F-12 Mix completed with 2% B27, 10 ng/ml epidermal growth factor, 20 ng/ml basic fibroblast growth factor, 3.3% FKS, 100 u/ml penicillin, 100 ug/ml streptomycin, 2 mmol/L L-glutamine).

Afterwards the medium with the detached cells was centrifuged for 5 minutes at 353 g, the supernatant discarded and the cells resuspended in IDH medium, and cultured in a total volume of 3 ml medium (IDH) in a 9.6 cm² culture container (conditions: 5% CO2, 37° C., 95% humidity).

Alternatively, a medium could be used containing 5% human serum instead of 3.3% FBS without B27. Alternatively, the commercially available medium Opti pro™ (Gibco, 12309) complemented with the corresponding amounts of serum, penicillin/streptomycin, 200 mM L-alanyl-L glutamine (Biochrom, K0302, 20 ml/L), EGF and FGF could be used.

The cells are passaged at a confluence of 70-90%. For this, the medium is removed, the cell layer is rinsed once with PBS, and the cells are trypsinized. Then, 5,000-6,000 cells are sown per cm² cell culture container surface. The cells or cell preparations according to the invention were isolated up to four times from the same explant at intervals of 6-10 days (depending on the biopsy material).

### Example 2

### Staining

The trypsinized cells were washed with PBS/0.5% BSA. Afterwards 250,000 cells were incubated on ice for 15 minutes in 0.1 ml PBS/0.5% BSA and the corresponding antibody (AK). Fluorescein isothiocyanate (FITC) labeled, R-phycoerythrin (PE) labeled and allophycocyanin (APC) labeled mouse anti-human AK were used (see Table X). The cells were washed with PBS/0.5% BSA after staining. Apoptotic cells were labeled with propidiumiodide (PI, Sigma, Taufkirchen, Germany), in order to exclude them from evaluation. The analysis was conducted using the FACSCalibur device (Becton Dickinson, Heidelberg, Germany) and the evaluation performed with help of CellQuest Software (Becton Dickinson).

TABLE 1
Information concerning antibodies used

| Antibody | Dilution | Manufacturer | Order No. |
|---|---|---|---|
| FITC α human CD90 | 1:75 | Pharmingen | 555595 |
| FITC α human CD105 | 1:20 | Acris | SM1177F |
| APC α HumanCD117 | 1:20 | Invitrogen | CD11705 |
| PE α humanCD166 | 1:20 | Pharmingen | 559263 |
| FITC α humanCD45 | 1:100 | Pharmingen | 555482 |
| PE α humanCD34 | 1:50 | Pharmingen | 555822 |
| PE α humanCD73 | 1:20 | Pharmingen | 550257 |

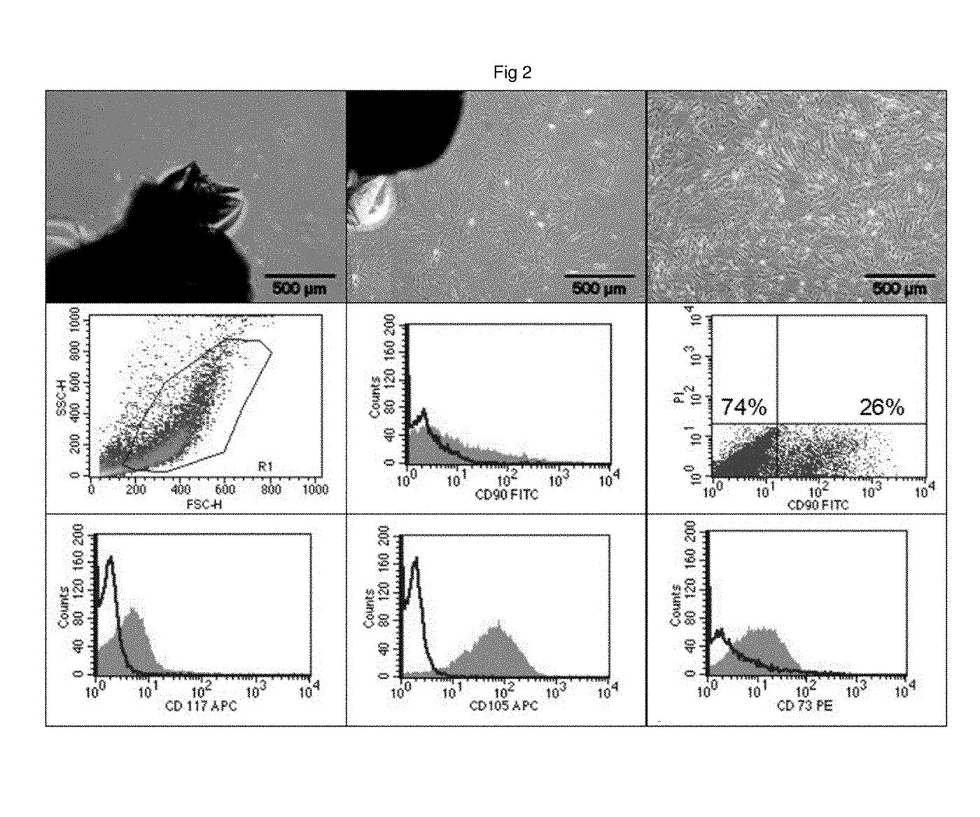

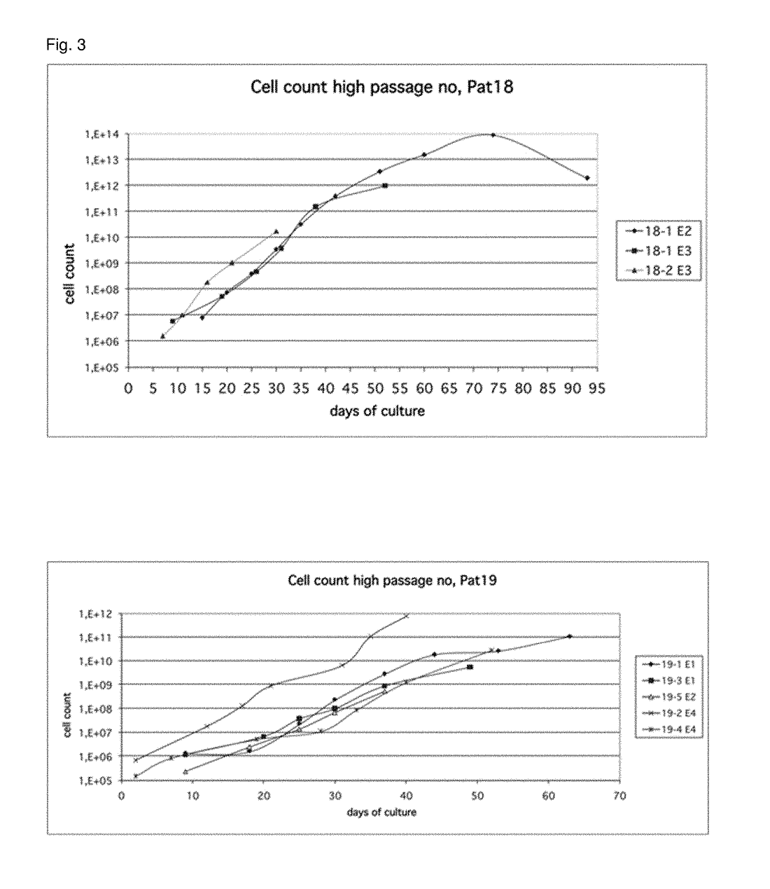

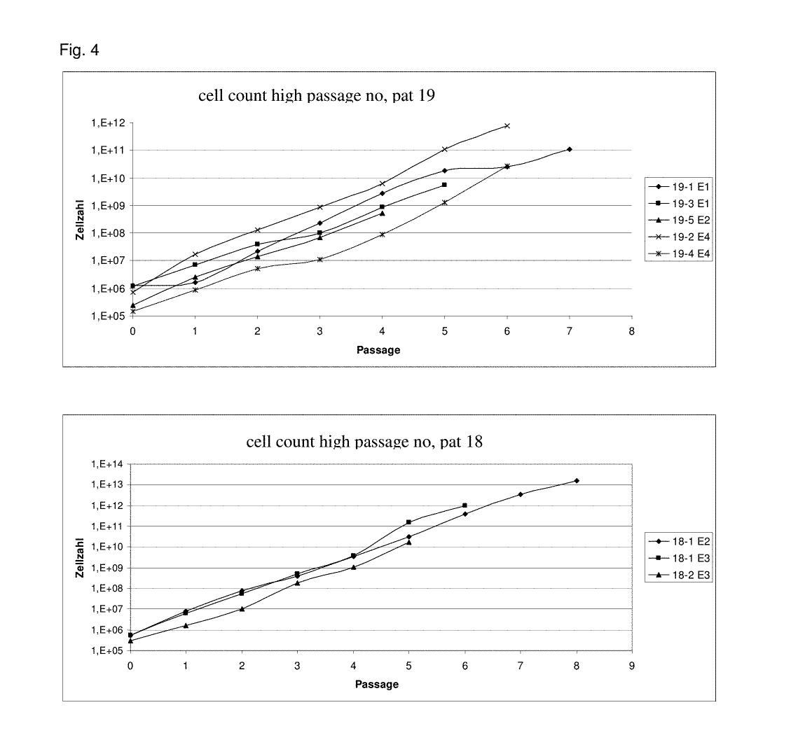

FIG. 2 shows in the top row, left and middle panels, adherent cells growing in culture from the biopsy material of (left: after 5 days in culture), which expand and adopt a fibroblast-like shape after some days (middle: after 15 days in culture). Top row, right panel shows a cell culture of the harvested cells in passage 4 after 5 days in culture. Middle and bottom rows, as labelled show in the FACS analysis, that cells are to the largest extent negative for CD90 and CD117, and positive for CD105 and to the largest extent also positive for CD73. The line shows the histogram of the negative (unstained) cell population, the histogram of the stained cells is depicted two-dimensionally.

The following Table 2 exemplifies parameter used in a measurement with the device “FACSCalibur”(Becton-Dickinson):

TABLE 2
Measurement parameters of the channels of the FACS device

| Param | Detector | Voltage | Amplification | Mode |
|---|---|---|---|---|
| P1 | forward scattering | E-1 | 3.27 | Lin |
| P2 | sidewards scattering | 424 | 1.00 | Lin |
| P3 | fluorescence chanel1 | 505 | 1.00 | Log |
| P4 | Fl. 2 | 489 | 1.00 | Log |
| P5 | Fl. 3 | 590 | 1.00 | Log |
| P6 | Fl. 2-A | | 1.00 | Lin |
| P7 | Fl. 4 | 740 | | Log |

TABLE 3
Compensations of the channels of the FACS device were
Compensation

FL1 - 2.5% FL2
FL2 - 1.0% FL1
FL2 - 2.0% FL3
FL3 - 14.6% FL2
FL3 - 0.8% FL4
FL4 - 1.0% FL3

Primary threshold parameter: Fluorescence channel 1, value: 35

### Example 3

### Culture with 5-azacytidine (Myogenic Induction, Myogenesis)

After stimulation with 5-azacytidine (24 h-20 μl/ml, 10 μM) and 4 week cultivation the cells were positive for α-desmin-antibodies and α-smooth muscle myosin-antibody. (according to: Xu W, Zhang Z, Mesenchymal stem cells from adult human bone marrow differentiate into a cardiomyocyte phenotype in vitro, Exp Biol Med (Maywood). 2004 Jul;229(7):623-31)

### Differentiation in Fat/Bones/Cartilage (“Multilineage”)

When cells were induced in accordance with the modified protocols of Pittenger et al. (Pittenger et al., Multilineage potential of adult human mesenchymal stem cells. Science. 1999 Apr 2;284(5411):143-7) for the cells described here, they did not differentiate in to fat, bone and cartilage. For this, the protocols of Pittenger et al. were adapted to the media used in example 1.

### Example 4

### Isolation of a Cell Population by Means of FACS Sorting

Cells were stained analogously to example 2, wherein: 10-15 million cells were labeled with αhumanCD90 FITC (BD) in a 1:100 dilution. The cells were washed with 10 ml PBS/BSA and prepared in a 1 ml PBS/BSA working volume.

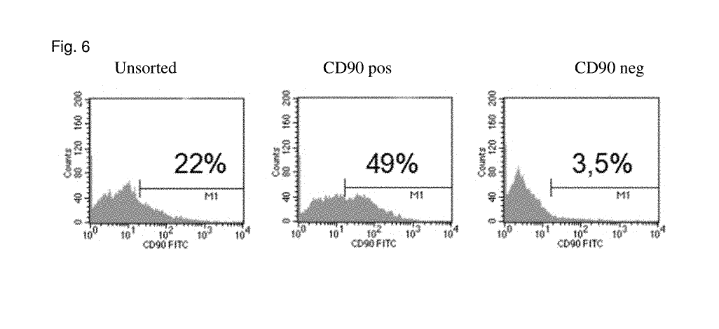

Cells stained in this way were sorted using known methods, for example by means of FACS sorting. The result of the sorting is shown in FIG. 6. The percentage values state the number of the CD90 positive cells. Before sorting 22% of the cells were CD90 positive, then, in the population sorted with respect to CD90, 49% of the cells were CD90 positive. The population sorted with respect to CD90 negative was 96.5% negative.

### Example 5

### Mixed Lymphocyte Reaction (MLR)

By means of the MLR, alloreactive T cells can be detected in the experiment. These are evidence that a rejection of alien tissue/alien cells will occur. In the MLR lymphocytes of an individual (donor A) are mixed with lymphocytes of another individual (donor B). When the T cells of the one individual recognize the MHC molecules of the other individual as alien, these T cells proliferate. The proliferation of donor A cells was prevented by means of treatment of the cells with mitomycin C (m), a cytostatic compound.

CardAP cells exhibit immunomodulatory properties. As the MLR shows, they prevent proliferation of T cells at numbers as low as 5×10⁴ cells, and thus act as immunosuppressants. Thus, these cells behave similar to mesenchymal stem cells concerning the MLR. This is important evidence for the possibility of an allogeneic application of the cells.

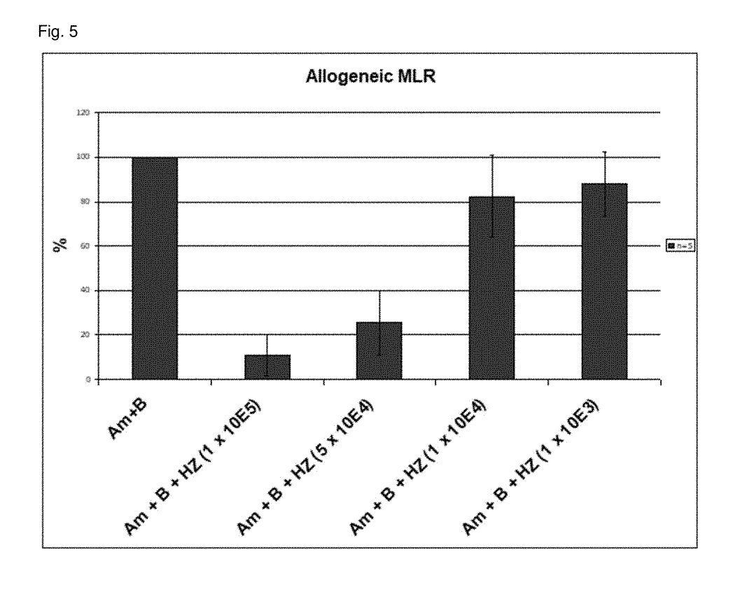

Different concentrations of heart cells (HZ) were mixed with mitomycin treated cells of donor A (Am). At a concentration of 5×10⁴ and 1×10⁵ heart cells, the proliferation and thus the immune reaction of the cells of donor B were suppressed. The immune reaction of the cells of donor A (mitomycin treated, Am) with the cells of donor B (see FIG. 5) served as a reference.

TABLE 4
Antibodies for the FACS analysis prior to MLR

| AB denotation | Company | Product number |
|---|---|---|
| CD31 - PE | BD | 555446 |
| CD34 - PE | BD | 550761 |
| CD45 - FITC | BD | 555482 |
| CD73 - PE | BD | 550257 |
| CD80 - PE | BD | 557227 |
| CD86 - PE | BD | 555653 |
| CD90 - FITC | BD | 555595 |
| CD105 - PE | Caltag | MHCD10504-4 |
| HLA I-FITC | BD | 555552 |
| HLA II-FITC | BD | 555558 |
| CD 40 PE | Serotec | MCA1590PE |

### Example 6

### Isolation of Atrial Appendage-derived Cells

Handling of atrial appendages turned out to be unproblematic. Preliminary tests and experiences with EMBs and atrial appendages indicated that most CD90− cells could be obtained from endomyocardial areas whereas CD90+ cells, which are most likely cardiac fibroblasts, would grow in epicardial areas. Therefore, the epicardium was removed from atrial appendages and discarded. Cutting fragments from the remaining myocardium and attaching them to the bottom of 6-well-plates was easily carried out. In the outgrowth cultures, cells with a fibroblast-like morphology first appeared after 6 to 8 days. Harvest was performed after 12 to 14 days when a ring of confluent cells surrounded the tissue fragments. Normally, CD90+ fibroblasts are present ubiquitously in the heart. Thus, to ensure that only a CD90^{low} cell population would be further cultured, cell sorting via MACS was performed using CD90-labelled magnetic microbeads. Flow cytometry analysis was performed before and after sorting. On average CD90 expression could be lowered from 68.66+/−4.43 SEM % to a mean expression of 18.51+/−5.62 SEM %.

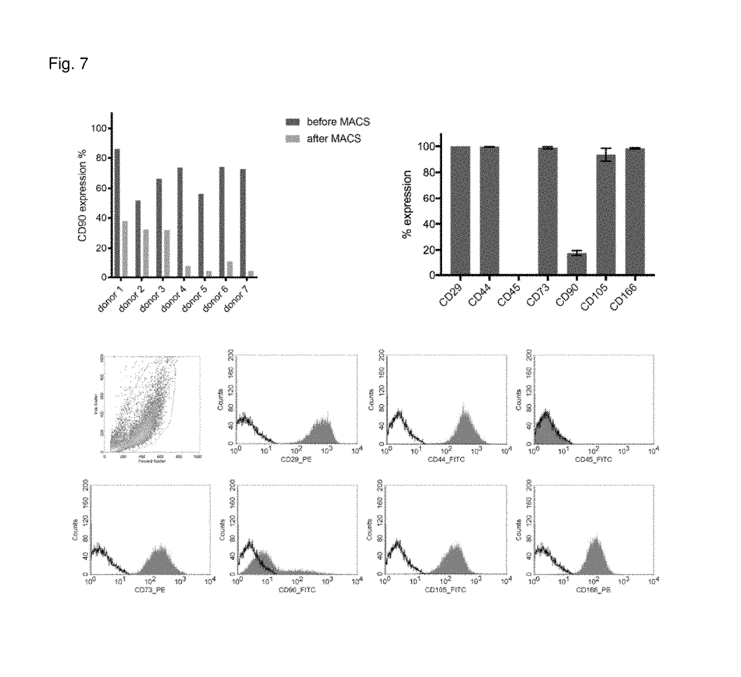

Individual values are illustrated in FIG. 7.

Although CD90^{low} atrial appendage-derived cells are not only defined by their predominant absence of CD90, this marker seems to play an important functional role. In studies with cardiosphere-derived cells, high CD90 expression was associated with lower efficacy in terms of scar size reduction in a myocardial infarction mouse model. Before, it was reported that expression of the stem cell marker c-kit was responsible for the positive effects of those cells.

### Example 7

### Atrial Appendage-derived Cells show Fibroblast-like Morphology Though they are Predominantly Negative for Fibroblast Marker CD90

To demonstrate culture homogeneity of sorted appendage-derived cells, immunofluorescence staining for CD90 was performed. Human dermal fibroblasts served as a positive control. Only sporadic staining with lower intensity of the fluorescent dye occurred in atrial appendage-derived cell populations, showing that they are characterized by a great part of CD90− cells (stained nucleus but unstained membrane) and a small fraction of CD90+ or CD90^{low} cells.

### Example 8

### Atrial Appendage-derived Cells Match the Defined Expression Profile for CardAP Cells

Flow cytometry analysis was performed for CD90^{low} atrial appendage-derived cells from 7 donors in passage 4 for a defined set of surface antigens. FIG. 7 shows that isolated and subsequently sorted and expanded cells are CD29+, CD44+, CD45−, CD73+, CD90^{low}, CD105+ and CD166+. Therefore, CD90^{low} atrial appendage-derived cells express certain markers characteristic for mesenchymal cells like fibroblasts, but obviously their origin is different from classical cardiac fibroblasts. This surface marker profile has also been reported for EMB-derived CardAP cells, indicating a commonality between CardAP cells and atrial appendage-derived cells.

### Example 9

### VEGF and IL-8 Secretion Shows Pro-angiogenic Potential of Atrial Appendage-derived Cells

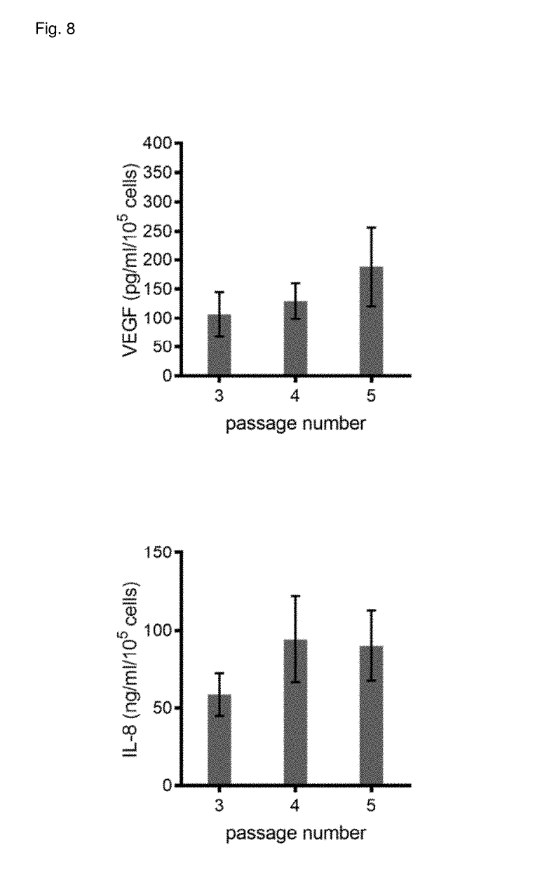

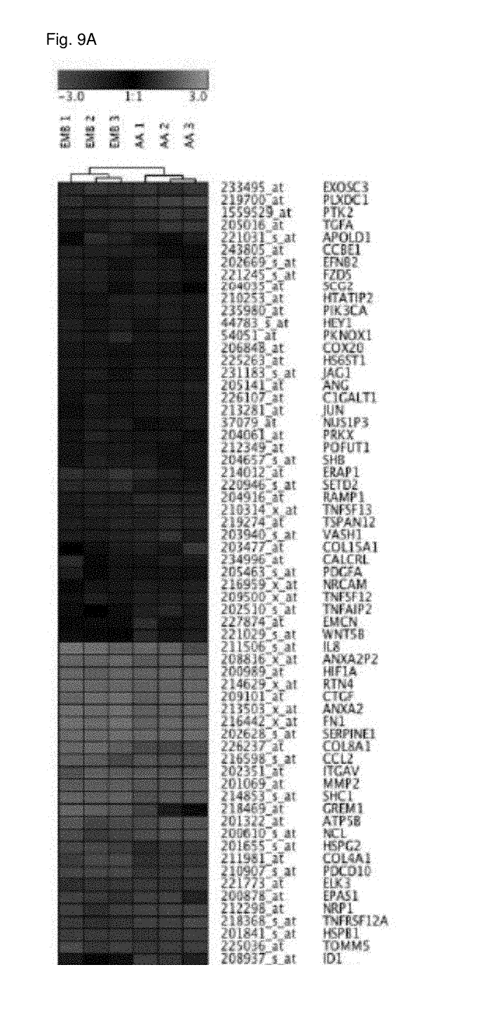

In passage 3, 4 and 5 CD90^{low} atrial appendage-derived cells were seeded and cultured. After 24 h medium supernatents were collected and later measured for proangiogenic factors VEGF and IL-8. VEGF values amounted to 47.14 (minimum) and 385.54 (maximum) pg/105 cells. IL-8 secretion was measured as 37.64 (minimum) and 169.90 (maximum) ng/105 cells. Mean expression values are shown in FIG. 8. An ordinary one-way ANOVA test showed no significant difference between passages. VEGF and IL-8 secretion was measured, because both factors play an important role in the formation of new blood vessels. VEGF induces angiogenesis and promotes neo-vascularization. They are both able to induce stem cell mobilization. Although cell-based therapies act through different mechanisms and depend on the respective cell type, one major effect of all current cell-based therapies for chronic heart diseases is described by the paracrine hypothesis, i.e. the secretion of paracrine factors such as chemokines and growth factors supporting cardiac repair by neoangiogenesis and myocardium regeneration.

Today, stem cells are the main cell source for cell-based therapy approaches for cardiac diseases like coronary artery disease or cardiomyopathy. CDCs for instance were believed to renew necrotic or fibrotic areas by generating new cardiomyocytes after application. More recent work shows that this hypothesis is questionable. Their positive effects on left ventricular ejection fraction and scar size reduction after infarction could rather derive from vasculogenesis. Therefore, CDCs may act similar to mesenchymal stem cells and other cells for cardiac cell therapy, i.e. based on paracrine effects. In fact, though stem cells have the ability to differentiate into cells of the target tissue, transdifferentiation of transplanted cells is controversial and the differentiation that occurs is unlikely to be connected to the improvement in the diseased heart. Whereas most of the studies with cell-based therapies were conducted with the aim to treat myocardial infarct patients, only a few approaches focused on cardiomyopathies such as DCM. While neovascularization as healing mechanism in the post-infarction heart appears rather obvious, DCM is also characterized by vascular dysfunction. Therefore, the proposed repair mechanisms for DCM patients consist not only in the reduction of fibrosis but also in angiogenesis.

This shows, that even though coronary artery disease/myocardial infarction and DCM have different pathomechanisms, angiogenesis is one of the key attempts in cell-based treatments and atrial appendage-derived cells show the potential to support this important healing process.

According to these findings, the inventors believe that facilitation of angiogenesis is one of the keys towards myocardium regeneration. The inventors recently showed that non stem cell EMB-derived cells, so called CardAP cells, have pro-angiogenic potential. Compared to these cells, atrial appendage-derived cells show similar mean expression values for IL-8, i.e. 94.35+/−27.78 SEM ng/105 cells in passage 4 compared to 98.62+/−17.18 SEM ng/105 cells in passage 5 for CardAP cells. VEGF maximum mean expression is three times lower in atrial appendage-derived cells: 188.47+/−67.58 SEM pg/105 cells in passage 5 compared to 641.24+/−200.70 SEM pg/105 cells (CardAP) in passage 6. But necessary secretion amounts for clinical applications have yet to be elucidated.

In a study with endothelial progenitor cells, IL-8 secretion of 33.3+/−5.8 SEM ng/105 cells and VEGF secretion as low as 53+/−10.3 SEM pg/105 were postulated to contribute to neovasculogenesis in mice in vivo. In another study, bone marrow mesenchymal stem cells supplemented with 200 pg/ml VEGF (˜2 ng VEGF/kg body weight per intramuscular injection) showed cardiac repair in failing hamster hearts.

### Example 10

### Microarray Data Mining Shows Pro-angiogenic Gene Profile

The inventors hypothesized that CD90low atrial appendage-derived cells express key genes involved in angiogenesis and vasculogenesis.

The inventors therefore screened the three datasets for expression of 84 key genes involved in angiogenesis. 60 out of 84 genes were present in all three datasets as at least one probe set ID (Table 1). As expected and also shown in immunosorbent assays on the protein level (FIG. 8), they express VEGF and IL-8, but also genes involved in the VEGF pathway such as kinase insert domain receptor (KDR), sphingosine kinase 1 (SPHK1) and V-akt murine thymoma viral oncogene homolog 1 (AKT1). The same screening procedure was undertaken for micro array data of CardAP cells, which are isolated form EMBs by the same protocol. CardAP cells have been shown to express many genes involved in angiogenesis such as VEGF, KDR, angiopoietin-1 and neuropilins.

This search also resulted in 60 present genes, though detection differed for 8 genes (Table 1), namely chemokine ligand 11 (CCL11), coagulation factor III (F3), fibroblast growth factor 1 (FGF1), fms-related tyrosine kinase 1 (FLT1), hepatocyte growth factor (HGF), midkine (MDK), transforming growth factor alpha (TGFA) and Tyrosine kinase with immunoglobulin-like and EGF-like domains 1 (TIE1).

Whereas FLT1, HGF, MDK and TIE1 were detected in CD90^{low} atrial appendage-derived cells, they were not in CardAP cells. On the other hand, CCL11, F3, FGF1 and TGFA could be detected in CardAP cells but not in CD90^{low} atrial appendage-derived cells. Although all of these genes are involved in angiogenesis, the way of involvement differs among genes. The FLT1 gene for example encodes for a VEGF receptor, which binds VEGFA, VEGFB and placental growth factor (PGF). It is involved in the regulation of angiogenesis and can promote endothelial cell proliferation, survival and angiogenesis. HGF acts as growth factor for different tissues. In animal studies, it was found upregulated after myocardial infarction. Its application can reduce infarct size and induce attraction of cardiac stem cells.

Though more than 70% of key angiogenesis genes could be detected as present, this micro array analysis can only be seen as first screening to get an overview and no complex bioinformatics or statistics were intended.

In the next step, the inventors looked further into differences in the angiogenic profile of both CD90^{low} atrial appendage-derived cells and CardAP cells from EMBs.

To compare the angiogenic expression profile of both cell types, every GeneChip of atrial appendage-derived cells from 3 donors was compared to every GeneChip of EMB-derived cells also from 3 donors, leading to 9 comparisons.

The inventors assumed significant differences for a ≤-2 decreased fold change or ≥2 induced fold change in expression, that occurred in at least 80% of the comparisons. The search resulted in 1031 differentially expressed probe sets which were higher expressed and 592 differentially expressed probe sets which were lower expressed in atrial appendage-derived cells compared to EMB-derived CardAP cells, representing 811 and respectively 398 genes.

To find out in which biological processes these genes are involved, probe sets were uploaded to DAVID and functionally clustered. The results were screened for key terms of cardiac regeneration, namely “angiogenesis”, “blood vessel development”, “blood vessel morphogenesis”, “cardiac muscle tissue development”, “cardiac muscle cell differentiation” and “vasculature development”.

The search resulted in 27 genes with higher expression values in atrial appendage-derived cells and 11 genes with higher expression in EMB-derived CardAP cells (Table 2 and 3).

Interestingly, among the genes with increased fold change are CXCL12, also known as stromal cell-derived factor 1α (SDF-1αa) and Wt1 (Wilms tumor 1). SDF-1α is involved in stem cell homing and recruitment of endothelial progenitor cells. It is believed to be one of the pivotal genes during regeneration of the vasculature. SDF-1α levels are upregulated after myocardial infarction, indicating its beneficial role in restoring damaged heart tissue. Wt1 is an epicardial progenitor marker which is also highly expressed after myocardial infarction, helping to re-vascularize the heart.

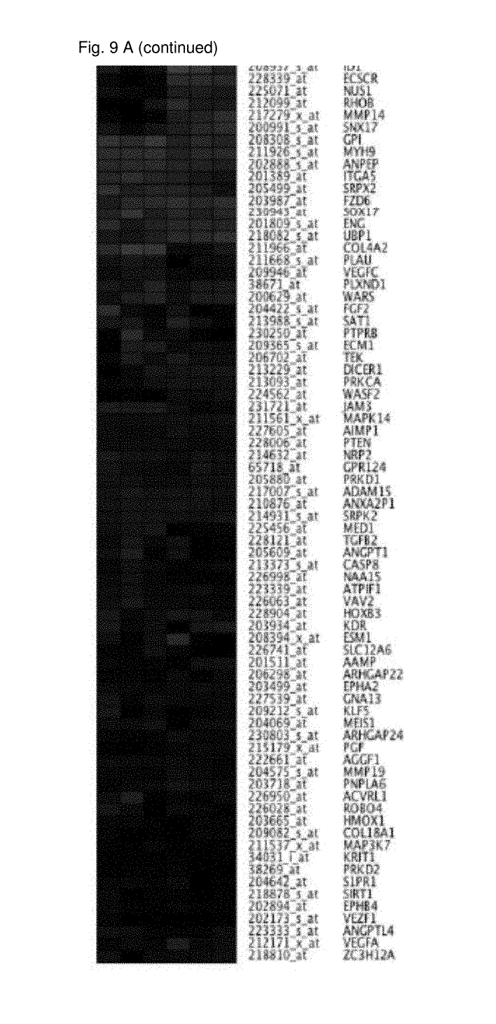

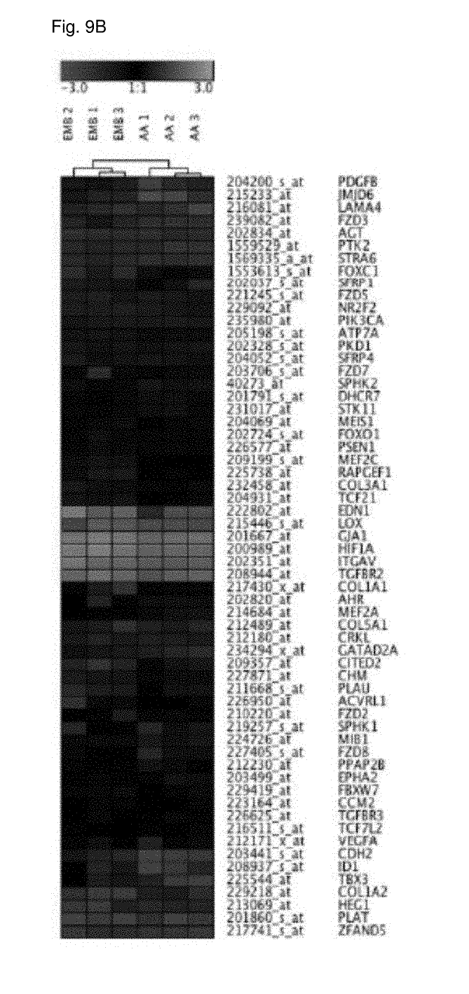

To elucidate if the increased expression of these genes goes beyond the mRNA level, further research needs to be conducted. Though differentially expression of certain genes could be shown, the inventors hypothesized that, in general, atrial appendage-derived cells and EMB-derived CardAP cells belong to the same cell family and share similar expression profiles. To undermine this hypothesis, hierarchical clustering was performed using GENESIS 1.7.7 software (Sturn et al., 2002). This time, all genes annotated with the terms “angiogenesis”, “blood vessel development”, “blood vessel morphogenesis” and “vasculature development” were included, provided that genes were expressed in all 6 gene chips and p-value was <0.05. For clustering, two groups were formed according to the GO terms assigned to the genes: “angiogenesis” (group 1) and “blood vessel development”, “blood vessel morphogenesis” and “vasculature development” (group 2). As shown in FIG. 9, EMB-derived CardAP cells and atrial appendage-derived cells clustered in two separate groups when clustered for experiments, which might be due to their different origin. However, genes were assembled in rather homogenous clusters, indicating strong similarities among both cells types. Taking these findings together, likewise cardioprotective CardAP cells, CD90^{low} atrial appendage-derived cells express a variety of pro-angiogenic genes. If they also share their anti-fibrotic, anti-hypertrophic and immune modulating features has to be elucidated by further studies.

### Example 11

### Evaluation of Growth Curves Shows Potential Suitability for use as Cell Product

To estimate how many CD90^{low} cells could be generated from a single atrial appendage, the mean growth rate of CD90low cells during the first 4 passages, the mean number of fragments of ˜1 mm3 into which one appendage could be divided, and the number of CD90^{low} cells that can be generated from these fragments by magnetic cell sorting and subsequent culture expansion was determined.

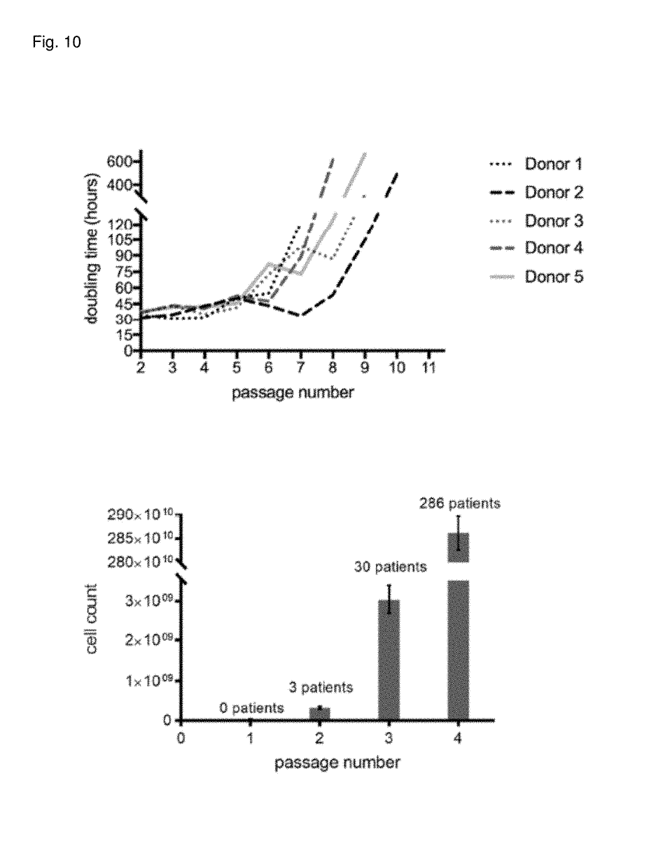

First, CD90^{low} cells from 5 different atrial appendages were grown up to passage 1. In passage 2 they were seeded at a density of 6 000 cells/cm2, cultured for 5-6 days, counted and plated again until the cell number was less then needed to plate them again. As shown in FIG. 10 for all 5 donors, the maximal passage number varied between 7 (donor 1) and 10 (donor 2). In passages 2 to 5 growths parameter for all 5 donors were similar with a mean doubling time td of 37.94 h and mean growth rate P of 0.01827/h. After seeing the rather steady growth during the first passages, the inventors wanted to calculate how many cells could be obtained from one appendage in total. The inventors therefore prepared atrial appendages from 3 different donors as described above and divided them into fragments of ˜1 mm3 which led to 243, 234 and 277 fragments (Table 4). For every donor, 126 fragments were fixed on 6 well plates, placing 3 fragments in 1 well. Cells were harvested after 13 days, pooled and then sorted via magnetic microbeads as described above. The results (Table 4) were used to calculate the mean number of CD90low cells that could be seeded in passage 1. Taking this number and using the growth rate P=0.01827/h, theoretical cell numbers in passage 3 and 4 could be calculated, which range from 2.72×109 to 3.42×109 in passage 3 and in passage 4 from 2.57×1010 to 3.23×1010.

In the presented study design for a phase I/II clinical trial on treatment of patients with DCM with autologous EMB-derived CardAP cells, patients are supposed to receive cell numbers ranging from 5×105 to 1×106 per kg of bodyweight for intravenous applications and between 26×106 and 113×106 cells for the intramyocardial application. Using 100×106 cells as basis for the calculations, in possible atrial appendage-based applications, from one atrial appendage therefore could be generated cell numbers that could serve more than 250 patients (FIG. 10). The number of injected cells in stem cell-based clinical studies varies between different trials and cell types. In the TOPCARE-DCM study for example, 33 patients were treated with 259+/−135×106 bone marrow mononuclear cells. Participants of the CADUCEUS trial on the other hand received only between 12.5×106 and 25×106 cells.

Fact is, in order to get the most benefit out of the delivered cells it is vital that they remain viable in the administered area of the heart. Only then they can take therapeutic effects. Hence it will not only be important to find the best matching cell type for a certain patient collective but also to find the best delivery method and the right number of cells injected. Poor cell retention remains one of the major problems in cardiac cell therapy and is due to wash-out, ischemia or inflammation (Robey et al., 2008). Approaches to solve these problems will be pivotal for the progress of cell-based therapies.

The idea of atrial appendage-derived cells is to offer an off-the-shelf product for an allogeneic application. As opposed to endomyocardial biopsies that are currently under investigation for an autologous treatment approach, atrial appendages offer a large tissue mass that allows for isolating several billion cells which can be stored in single dosages for later application. EMB-derived CardAP cells that are isolated by the same protocol have already shown low immunogenicity, a necessary characteristic for an allogenic therapy, that is currently being investigated for CD90^{low} atrial appendage-derived cells. It should also be considered to inject the cells at defined points of time, e.g. once a year. As for skeletal myoblasts, repeated injection of cells led to a better outcome. Clinical trials with CardAP cells will show for how long beneficial outcomes remain and if a repeated application will be reasonable or even necessary.

### CONCLUSION

Atrial appendage-derived cells are easy to isolate and expand, and secrete proangiogenic VEGF and IL-8. Their similarity to endomyocardial biopsy-derived CardAP cells could imply that they also carry the same beneficial characteristics with regard to cardioprotective effects and low immunogenicity. The latter is under current investigation for atrial appendage-derived cells. Altogether, the atrial appendage represents a promising source for cardiac-derived cells for allogenic cell-based therapy of chronic cardiac diseases.

While the invention has been described in terms of various preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

We claim:

1. A method for obtaining a cell preparation comprising isolated fibroblast-like cells, the method comprising:
a) in a first cell culture step, culturing a tissue sample obtained from mammalian heart muscle tissue under conditions suitable for culture of mammalian cells in cell culture medium, in a cell culture container having a solid surface;
b) detaching cells that have grown out of the tissue sample and that adhere to the solid surface in a passage step by means of limited proteolysis, isolating the detached cells;
c) culturing the sorted cells in a second cell culture step;
thereby obtaining the cell preparation that is CD90 negative; CD105 positive; CD106 negative and CD117 negative.

2. The method according to claim 1, wherein said fibroblast-like cells are characterized by
a) an expression of one or more genes selected from ACTC1, POP3, VE-Cadherin (CDH5) and CDH13 and/or
b) a lack of expression of NPNT and/or TERT.

3. The method according to claim 2, wherein said fibroblast-like cells are characterized by expression of ACTC1, POP3, VE-Cadherin (CDH5) and CDH13 and by lack of expression of NPNT and TERT.

4. The method according to claim 1, wherein said tissue sample comprises or essentially consists of endomyocardial cells.

5. The method according to claim 1, wherein said tissue sample comprises or essentially consists of atrial appendage cells, and subsequent to said passage step, CD90 positive cells are excluded from the detached cells via magnetic activated cell sorting or fluorescence activated cell sorting in a CD90-sorting step.

6. The method of claim 1, wherein the tissue sample is digested, prior to the first cell culture step, by one or more connective tissue digesting enzymes

7. The method of claim 6, wherein the connective tissue digesting enzyme comprises at least one of trypsin-EDTA and collagenase IV, and wherein the time limited digestion is less than 10 minutes at an activity of 0.05 to 0.25 u/500 ml for trypsin and/or 0.2 to 4.5 u/ml for collagenase IV.

8. The method of claim 1, wherein the cell culture medium of either the first cell culture step, the passage step or of both steps does not contain any of cardiotrophin, thrombin and mercaptoethanol.

9. The method of claim 1, wherein the first cell culture step (a) has a duration of 7 to 15 days.

10. The method of claim 1, wherein the cells are maintained in said second cell culture step for a period of 7 to 15 days or 7 to 12 days.

11. The method of claim 1, wherein the passage step (b) is conducted once the cells adhering to the solid surface are at least 70% confluent.

12. A cell preparation produced by the method according to claim 1.

13. The cell preparation according to claim 12, wherein the cells contained in the cell preparation are more than 90% CD90 negative, more than 90% CD105 positive, and more than 50% CD117 negative.

14. The cell preparation according to claim 12, wherein the cells contained in the cell preparation are more than 95% CD90 negative, more than 95% CD105 positive, more than 60% CD117 negative, and more than 50% CD166 positive.

15. The cell preparation according to claim 12, wherein the cells contained in the cell preparation are more than 90% CD34 negative and more than 90% CD45 negative.

16. The cell preparation according to claim 12, wherein the cells contained in the cell preparation are more than 95% CD90 negative, and the CD90 negative portion of the cell preparation is more than 90% CD105 positive.

17. The cell preparation according to claim 12, wherein the cells contained in the cell preparation are more than 95% CD90 negative, and the CD90 negative portion of the cell preparation is more than 90% CD105 positive and more than 50% CD117 negative.

18. The cell preparation according to claim 12, wherein the cells contained in the cell preparation are more than 98% CD90 negative, and the CD90 negative portion of the cell preparation is more than 95% CD105 positive, more than 60% CD117 negative and more than 50% CD166 positive.

19. The cell preparation according to claim 12, wherein the cells contained in the cell preparation are more than 95% CD90 negative, the CD90 negative portion of the cell population is more than 90% CD105 positive and the portion of the cell preparation being CD90 negative and CD105 positive at the same time is more than 60% CD117 negative.

20. The cell preparation according to claim 12, wherein the cells contained in the cell preparation are more than 90% negative for CD106 (VCAM1) and/or the cells contained in the cell preparation express ACTC1 and/or the cells contained in the cell preparation express POP3 and/or the cells contained in the cell preparation express POP3 and/or the cells contained in the cell preparation express VE-Cadherin (CDH5) and/or the cells contained in the cell preparation express CDH13 and/or the cells contained in the cell preparation do not express NPNT and/or the cells contained in the cell preparation do not express TERT.

21. A method for treatment of cardiac disease, comprising
administering to a subject in need thereof a therapeutically effective amount of a cell preparation comprising isolated fibroblast-like cells, obtained from mammalian heart muscle tissue, wherein the isolated fibroblast-like cells are CD90 negative; CD105 positive; CD106 negative and CD117 negative.
